SEQUENCE LISTING (53 sequences)

SEQ ID NO: 1 - protein, 142 aa, Mannheimia haemolytica
MRKLLVITALTLCTTPVFAADKNVIFSCTSTEGKPLTVKRVGNDYEYSYDKTTFKNPIKKAVTNDGSIIARGSGFTTYALELENDGLKYLVGFVQPNGNAKEFIEPGATISQSKEQPSIGSVDCDTRKKSHYKFDVHLMNTL

SEQ ID NO: 2 - protein, 123 aa, Mannheimia haemolytica
ADKNVIFSCTSTEGKPLTVKRVGNDYEYSYDKTTFKNPIKKAVTNDGSIIARGSGFTTYALELENDGLKYLVGFVQPNGNAKEFIEPGATISQSKEQPSIGSVDCDTRKKSHYKFDVHLMNTL

SEQ ID NO: 3 - DNA, 429 bp, Mannheimia haemolytica
atgcgtaaattattagtaattactgctcttacactctgcacgaccccagtatttgcagcagataaaaacgtaatattttcctgtaccagcacggagggaaaaccgcttactgttaaacgggtaggaaatgattacgaatattcgtatgacaaaacaaccttcaaaaatccgattaaaaaggcagtcactaatgacggctcaattatcgccagaggttcaggttttacaacgtatgcattagagctggaaaatgatggattgaagtatttggtcggctttgttcagccaaatggcaatgcaaaagaatttattgagccgggagcaacgattagccaaagtaaagaacaaccaagtatcggctctgttgattgtgatactcgtaaaaaaagccattataagtttgatgttcatttgatgaatactttataa

SEQ ID NO: 4 - DNA, 375 bp, Mannheimia haemolytica
atggcagataaaaacgtaatattttcctgtaccagcacggagggaaaaccgcttactgttaaacgggtaggaaatgattacgaatattcgtatgacaaaacaaccttcaaaaatccgattaaaaaggcagtcactaatgacggctcaattatcgccagaggttcaggttttacaacgtatgcattagagctggaaaatgatggattgaagtatttggtcggctttgttcagccaaatggcaatgcaaaagaatttattgagccgggagcaacgattagccaaagtaaagaacaaccaagtatcggctctgttgattgtgatactcgtaaaaaaagccattataagtttgatgttcatttgatgaatactttataa

SEQ ID NO: 5 - protein, 143 aa, Mannheimia haemolytica
MKKMAIVALSAFFSMNAFANNNVVYSCTTTDNQTLKVTKEGGNYVYSHGNTTFKNPVKEALKNPASEIAGGSQFTTISLELRNAGKSYIVGHIEADPKSPFEASVQIQDIKTGNDITSFECRSDKPIRHNFDRKLMRKSGFAA

SEQ ID NO: 6 - protein, 124 aa, Mannheimia haemolytica
NNNVVYSCTTTDNQTLKVTKEGGNYVYSHGNTTFKNPVKEALKNPASEIAGGSQFTTISLELRNAGKSYIVGHIEADPKSPFEASVQIQDIKTGNDITSFECRSDKPIRHNFDRKLMRKSGFAA

SEQ ID NO: 7 - DNA, 432 bp, Mannheimia haemolytica
atgaaaaaaatggcaattgtagctttatcagcatttttctcaatgaatgcttttgcaaataacaatgtagtgtattcttgcacaactacagataaccaaacgttaaaagttaccaaagaggggggaaactatgtgtactctcacggtaataccacattcaaaaatcctgtaaaagaagctctcaaaaatccggcatcggagattgcaggtggttcacaattcacaacaatctcgctcgaattaagaaatgcaggaaaatcttatattgtaggtcatatagaagcagaccctaaatcaccttttgaagcaagtgttcagattcaggatattaaaacaggaaatgatattacatcatttgaatgtcgttccgataagcctattcgccacaactttgatcgcaaactgatgagaaaatctggatttgcagcctag

SEQ ID NO: 8 - DNA, 375 bp, Mannheimia haemolytica
atgaacaatgtagtgtattcttgcacaactacagataaccaaacgttaaaagttaccaaagaggggggaaactatgtgtactctcacggtaataccacattcaaaaatcctgtaaaagaagctctcaaaaatccggcatcggagattgcaggtggttcacaattcacaacaatctcgctcgaattaagaaatgcaggaaaatcttatattgtaggtcatatagaagcagaccctaaatcaccttttgaagcaagtgttcagattcaggatattaaaacaggaaatgatattacatcatttgaatgtcgttccgataagcctattcgccacaactttgatcgcaaactgatgagaaaatctggatttgcagcctag

SEQ ID NO: 9 - protein, 137 aa, Actinobacillus pleuropneumoniae
MRKLTTLAVCSFISLPVLAENISIFSCTAEDGNQILIQKTGADYQFSYGKILFKNPAKQVLKNEGSYVATGSGFTTSALEMKSKSHSYTIEFVQPRGTNVIDSPMLYITKGNNTTEIKCDTNKKVGQHFEYSKMNIL

SEQ ID NO: 10 - protein, 118 aa, Actinobacillus pleuropneumoniae
ENISIFSCTAEDGNQILIQKTGADYQFSYGKILFKNPAKQVLKNEGSYVATGSGFTTSALEMKSKSHSYTIEFVQPRGTNVIDSPMLYITKGNNTTEIKCDTNKKVGQHFEYSKMNIL

SEQ ID NO: 11 - DNA, 414 bp, Actinobacillus pleuropneumoniae
atgcgtaaattaacaacattagcagtatgctcgtttatatctttaccggttttagcagaaaatatctcaattttttcttgtactgcagaagacggtaatcaaattctgatccaaaaaactggcgcagactatcaattttcatatgggaaaatcttatttaagaatccagctaaacaagtactgaaaaacgaaggttcttatgtggcgacaggctctggttttacaacaagtgccttagaaatgaaaagtaagagtcattcttatactattgagttcgtacaacctagaggaaccaatgtaattgactcaccaatgttgtatattacaaaaggtaataatacaactgaaataaaatgtgataccaataaaaaagttggacaacattttgaatacagtaaaatgaacattctataa

SEQ ID NO: 12 - DNA, 360 bp, Actinobacillus pleuropneumoniae
atggaaaatatctcaattttttcttgtactgcagaagacggtaatcaaattctgatccaaaaaactggcgcagactatcaattttcatatgggaaaatcttatttaagaatccagctaaacaagtactgaaaaacgaaggttcttatgtggcgacaggctctggttttacaacaagtgccttagaaatgaaaagtaagagtcattcttatactattgagttcgtacaacctagaggaaccaatgtaattgactcaccaatgttgtatattacaaaaggtaataatacaactgaaataaaatgtgataccaataaaaaagttggacaacattttgaatacagtaaaatgaacattctataa

SEQ ID NO: 13 - protein, 154 aa, Pasteurella dagmatis
MKKHILFTNHPKYKECPMKKITLITLLFFVSAATANNVVEDIDYQQEEPIFYCKAPNKKIIQLTKYEEVYIYRFGKNLDKPEIRLENHIEDVKRTSETQNGFETKRAYELTTGPYIYRIFSGIPKVPEENGVEVFKHRKRIARIECSGYVHIFE

SEQ ID NO: 14 - protein, 119 aa, Pasteurella dagmatis
NNVVEDIDYQQEEPIFYCKAPNKKIIQLTKYEEVYIYRFGKNLDKPEIRLENHIEDVKRTSETQNGFETKRAYELTTGPYIYRIFSGIPKVPEENGVEVFKHRKRIARIECSGYVHIFE

SEQ ID NO: 15 - DNA, 465 bp, Pasteurella dagmatis
atgaaaaagcatatactatttacaaatcatccaaaatataaggaatgccctatgaaaaaaataacactcattaccttattattctttgttagtgctgctacagccaataatgttgtagaagatatagactatcaacaagaagaacccattttctactgtaaagcgcctaacaaaaaaattattcaattaaccaaatatgaggaagtctatatttatcgtttcggtaaaaaccttgataaaccagagattagactcgaaaatcatattgaagatgtaaaacgaaccagtgaaactcaaaacggatttgaaactaaacgtgcctatgagttaacaactgggccttatatttatcgtatattttctggtattcctaaagttcctgaagagaatggtgttgaagtattcaaacatagaaaacgaattgcaaggatcgaatgttctggatatgtccatatttttgagtga

SEQ ID NO: 16 - DNA, 363 bp, Pasteurella dagmatis
atgaataatgttgtagaagatatagactatcaacaagaagaacccattttctactgtaaagcgcctaacaaaaaaattattcaattaaccaaatatgaggaagtctatatttatcgtttcggtaaaaaccttgataaaccagagattagactcgaaaatcatattgaagatgtaaaacgaaccagtgaaactcaaaacggatttgaaactaaacgtgcctatgagttaacaactgggccttatatttatcgtatattttctggtattcctaaagttcctgaagagaatggtgttgaagtattcaaacatagaaaacgaattgcaaggatcgaatgttctggatatgtccatatttttgagtga

SEQ ID NO: 17 - protein, 130 aa, Pasteurella multocida
MKSVYFFLPFVCASFSLNAANTSIPMLDKSYQESSQQQEEPIFTCRTNQGKIIQLNKYDKVYIYRFGANLNNPELTLTSHIENIIKGVITKGAIEIKRFYELTSGNYTYHIFYTNPKAYDENGAEVFKNN

SEQ ID NO: 18 - protein, 111 aa, Pasteurella multocida
ANTSIPMLDKSYQESSQQQEEPIFTCRTNQGKIIQLNKYDKVYIYRFGANLNNPELTLTSHIENIIKGVITKGAIEIKRFYELTSGNYTYHIFYTNPKAYDENGAEVFKNN

SEQ ID NO: 19 - DNA, 393 bp, Pasteurella multocida
atgaagtcagtatattttttccttccattcgtctgtgccagcttttctcttaatgccgctaatacaagtattcctatgttggataaatcttatcaggaatcttctcaacaacaagaagaacccatctttacttgcagaacaaatcaaggaaaaatcattcaattaaacaaatatgataaagtctatatctatcgctttggtgcaaatcttaataatccagaacttacactaacaagtcatattgaaaacattataaaaggggttatcacgaaaggcgcaatcgaaataaaacgtttctacgagctaacaagtggtaattatacgtatcatattttttataccaatcctaaagcttatgatgaaaatggtgcggaagtatttaaaaataactaa

SEQ ID NO: 20 - DNA, 339 bp, Pasteurella multocida
atggctaatacaagtattcctatgttggataaatcttatcaggaatcttctcaacaacaagaagaacccatctttacttgcagaacaaatcaaggaaaaatcattcaattaaacaaatatgataaagtctatatctatcgctttggtgcaaatcttaataatccagaacttacactaacaagtcatattgaaaacattataaaaggggttatcacgaaaggcgcaatcgaaataaaacgtttctacgagctaacaagtggtaattatacgtatcatattttttataccaatcctaaagcttatgatgaaaatggtgcggaagtatttaaaaataactaa

SEQ ID NO: 21 - protein, 142 aa, Haemophilus somnus
MRKLLGITVLTLCTTSAFATDKNVVFSCTSTEGKPLTVKRVGNDYEYSYDKTIFKNPIKKAVTNDGSIIARGSGFTTYALELKNDGLKYVVGFVQPNGNSKEFIEPGATISRDNDQPSIGSVDCDPYKKIYYKFDVHLMNTL

SEQ ID NO: 22 - protein, 123 aa, Haemophilus somnus
TDKNVVFSCTSTEGKPLTVKRVGNDYEYSYDKTIFKNPIKKAVTNDGSIIARGSGFTTYALELKNDGLKYVVGFVQPNGNSKEFIEPGATISRDNDQPSIGSVDCDPYKKIYYKFDVHLMNTL

SEQ ID NO: 23 - DNA, 429 bp, Haemophilus somnus
atgcgtaaattattaggtattactgtacttacactttgtacaacttcagcatttgcaacggataaaaatgtggtattttcttgtaccagcacagaaggtaaacctctcacagttaaacgggtaggaaatgattacgagtattcgtatgataaaactatcttcaaaaacccaatcaaaaaagcggtgaccaacgatggctcaattatcgctagaggatcaggttttaccacgtatgcattagaactgaaaaatgatggtttgaagtatgtagttgggtttgttcaaccaaatggtaactcaaaagagtttattgagcctggagcaacgattagccgtgataatgatcaaccaagcattggctctgtagattgtgatccttacaaaaaaatctattataagtttgatgttcatttaatgaataccttgtaa

SEQ ID NO: 24 - DNA, 375 bp, Haemophilus somnus
atgacggataaaaatgtggtattttcttgtaccagcacagaaggtaaacctctcacagttaaacgggtaggaaatgattacgagtattcgtatgataaaactatcttcaaaaacccaatcaaaaaagcggtgaccaacgatggctcaattatcgctagaggatcaggttttaccacgtatgcattagaactgaaaaatgatggtttgaagtatgtagttgggtttgttcaaccaaatggtaactcaaaagagtttattgagcctggagcaacgattagccgtgataatgatcaaccaagcattggctctgtagattgtgatccttacaaaaaaatctattataagtttgatgttcatttaatgaataccttgtaa

SEQ ID NO: 25 - protein, 164 aa, Edwardsiella ictaluri
MGRVIRIGRYGGLVSLPISGVQTAADPLLFQCGLDNGMVARLTLYDGVPRYGYGVMKHGRIYSPPLTLPRQEGERAMVRCGRSVLPGATYCRFINVNYRYVVMDSIGHDRSFTGVRVFHERKLVSPLNCRNGGFVISDYHAQGLIRDTDTGDEGAYGYLLPDAG

SEQ ID NO: 26 - protein, 140 aa, Edwardsiella ictaluri
ADPLLFQCGLDNGMVARLTLYDGVPRYGYGVMKHGRIYSPPLTLPRQEGERAMVRCGRSVLPGATYCRFINVNYRYVVMDSIGHDRSFTGVRVFHERKLVSPLNCRNGGFVISDYHAQGLIRDTDTGDEGAYGYLLPDAG

SEQ ID NO: 27 - DNA, 495 bp, Edwardsiella ictaluri
atgggcagggtaatccgtatcgggcggtatggggggctggtcagcctgccgatatcgggagtacagacggcagcggatccgctgctgttccagtgcgggctcgacaatggcatggtggcgaggctgaccctgtatgacggtgtgccgcgctatggctatggcgttatgaaacacggccgcatctactccccaccgctgacattgccgcgccaggagggtgagcgggccatggtgcgctgtgggagatcagttttgcccggggccacctattgccgttttattaatgtcaattatcgctatgtggtgatggatagcatcggccatgaccggtcatttaccggtgtccgggtgtttcatgagcggaagctcgttagcccccttaactgccgtaacggcggttttgttatcagtgattatcatgcgcaagggcttatccgggatacggacaccggagatgagggggcatatggctatcttttacccgatgccggctga

SEQ ID NO: 28 - DNA, 426 bp, Edwardsiella ictaluri
atggcggatccgctgctgttccagtgcgggctcgacaatggcatggtggcgaggctgaccctgtatgacggtgtgccgcgctatggctatggcgttatgaaacacggccgcatctactccccaccgctgacattgccgcgccaggagggtgagcgggccatggtgcgctgtgggagatcagttttgcccggggccacctattgccgttttattaatgtcaattatcgctatgtggtgatggatagcatcggccatgaccggtcatttaccggtgtccgggtgtttcatgagcggaagctcgttagcccccttaactgccgtaacggcggttttgttatcagtgattatcatgcgcaagggcttatccgggatacggacaccggagatgagggggcatatggctatcttttacccgatgccggctga

SEQ ID NO: 29 - protein, 145 aa, Haemophilus somnus
MRHKKLLFSTLAMVTFLTIMPVQAATKPIFSCSTAQGKKLLVEKTGSNYVLSYQDLKVTNPISQILERTNSHIVSRSGYVLYSLEFSDPKYSYYMQYQESMGDEKPLFAGLFRVEPNSNNAPTTLVKCNLKKPISNNFEINLMNN

SEQ ID NO: 30 - protein, 121 aa, Haemophilus somnus
ATKPIFSCSTAQGKKLLVEKTGSNYVLSYQDLKVTNPISQILERTNSHIVSRSGYVLYSLEFSDPKYSYYMQYQESMGDEKPLFAGLFRVEPNSNNAPTTLVKCNLKKPISNNFEINLMNN

SEQ ID NO: 31 - DNA, 438 bp, Haemophilus somnus
atgagacataaaaaactacttttttcaacattagcaatggtcacatttctcacgataatgcccgttcaagctgcaacaaaacctattttttcctgctctaccgcacaagggaaaaaacttttagtcgaaaaaacggggagtaattatgtattatcttatcaagatctcaaagtaactaaccccataagtcaaatattggagagaacgaattctcatatcgtgagccgttcaggctatgtgttgtattcacttgaattcagcgatccgaagtatagttattatatgcaataccaagaatcaatgggcgatgaaaaaccactctttgcagggcttttccgagttgaaccaaacagcaataatgccccaacaacactggtaaaatgtaacttgaaaaaaccgatttcaaacaattttgaaattaatttaatgaataactaa

SEQ ID NO: 32 - DNA, 369 bp, Haemophilus somnus
atggcaacaaaacctattttttcctgctctaccgcacaagggaaaaaacttttagtcgaaaaaacggggagtaattatgtattatcttatcaagatctcaaagtaactaaccccataagtcaaatattggagagaacgaattctcatatcgtgagccgttcaggctatgtgttgtattcacttgaattcagcgatccgaagtatagttattatatgcaataccaagaatcaatgggcgatgaaaaaccactctttgcagggcttttccgagttgaaccaaacagcaataatgccccaacaacactggtaaaatgtaacttgaaaaaaccgatttcaaacaattttgaaattaatttaatgaataactaa

SEQ ID NO: 33 - protein, 148 aa, Salmonella enterica
MHHYKFFALAAMLLCGSSFASDEYFMCNTAKGTIKLDENKGVLRYTLSKDRKTAFNYESKGNDYSGFKYNHYSRFQTDYFSVSFVNAEYKYTIFSNYEGESESRGVSVTNLNSKKESVYDCKSVSIDRLSDLSAKLACDKDSALGCEE

SEQ ID NO: 34 - DNA, 447 bp, Salmonella enterica
atgcatcattataagttttttgcgttggctgcaatgcttttgtgtggttcatcgtttgcgagtgatgaatatttcatgtgcaacactgcaaaaggaacaatcaaactggatgaaaacaaaggcgtgcttcggtacaccttatcgaaagaccgcaaaactgcatttaattatgaatcgaaaggtaatgattattcaggatttaaatacaatcattactcacgatttcaaaccgattatttcagtgtatcgtttgtaaatgcagagtataagtacacgatctttagtaattatgagggtgagagtgaaagccgtggagttagtgttacgaacctgaatagcaaaaaggaatctgtttacgattgtaagtccgttagcattgatcgtcttagtgatttgtcggcaaaactggcttgtgataaagattctgctttggggtgtgaagaatga

SEQ ID NO: 35 - DNA, 483 bp, Salmonella enterica
gaattctaccgaggacttaattaaataatgcatcattataagttttttgcgttggctgcaatgcttttgtgtggttcatcgtttgcgagtgatgaatatttcatgtgcaacactgcaaaaggaacaatcaaactggatgaaaacaaaggcgtgcttcggtacaccttatcgaaagaccgcaaaactgcatttaattatgaatcgaaaggtaatgattattcaggatttaaatacaatcattactcacgatttcaaaccgattatttcagtgtatcgtttgtaaatgcagagtataagtacacgatctttagtaattatgagggtgagagtgaaagccgtggagttagtgttacgaacctgaatagcaaaaaggaatctgtttacgattgtaagtccgttagcattgatcgtcttagtgatttgtcggcaaaactggcttgtgataaagattctgctttggggtgtgaagaatgataaaagctt

SEQ ID NO: 36 - protein, 163 aa, Salmonella enterica
MHHYKFFALAAMLLCGSSFASDEYFMCNTAKGTIKLDENKGVLRYTLSKDRKTAFNYESKGNDYSGFKYNHYSRFQTDYFSVSFVNAEYKYTIFSNYEGESESRGVSVTNLNSKKESVYDCKSVSIDRLSDLSAKLACDKDSALGCEEGILQDDDDKHHHHHH

SEQ ID NO: 37 - DNA, 519 bp, Salmonella enterica
gaattctaccgaggacttaattaaataatgcatcattataagttttttgcgttggctgcaatgcttttgtgtggttcatcgtttgcgagtgatgaatatttcatgtgcaacactgcaaaaggaacaatcaaactggatgaaaacaaaggcgtgcttcggtacaccttatcgaaagaccgcaaaactgcatttaattatgaatcgaaaggtaatgattattcaggatttaaatacaatcattactcacgatttcaaaccgattatttcagtgtatcgtttgtaaatgcagagtataagtacacgatctttagtaattatgagggtgagagtgaaagccgtggagttagtgttacgaacctgaatagcaaaaaggaatctgtttacgattgtaagtccgttagcattgatcgtcttagtgatttgtcggcaaaactggcttgtgataaagattctgctttggggtgtgaagaagggatcctgcaggatgacgatgacaaacaccatcatcaccatcattag

SEQ ID NO: 38 - protein, 128 aa, Avibacterium paragallinarum
MKKYLGLFLLAGLCSSPLLAEDYIVFSCFTAKGKQIALIESEGYYEYSFGKPGKPELVFKNKIADVEARSEKWNGVGSYWEAYNLKNGQYYYVVYKSENRVSDEHEFSGGVTVSKGDKEIATVSCERF

SEQ ID NO: 39 - DNA, 387 bp, Avibacterium paragallinarum
atgaaaaaatatctagggctatttctgcttgcagggctttgttcttcaccgctcttggcagaagattatattgtgttttcttgttttacggcgaaaggtaagcaaattgccttaatagaaagcgagggatattatgagtacagttttggcaaaccaggcaagcccgagttagtttttaaaaacaaaattgctgatgttgaagcgcgttctgaaaaatggaacggcgtaggatcttattgggaagcctataacctcaaaaatgggcaatattattatgtggtgtataaatcggaaaaccgtgtgagcgatgaacatgagttcagcggaggggtaacggtttctaaaggcgataaagaaatcgcaacggtttcttgcgaacggttttaa

SEQ ID NO: 40 - protein, 153 aa, Vibrio ichthyoenteri
MPLGFALLPLTAVAESLCPTTEQAVFSCEIGTKAVAACVAEDGKVSYRYGTQTKLELQLDEPVLSTGGCSGGGTSRLRFANGDYSYIVYDVMCNAEKIGPAQWSKTDYAGLMVLKGNKLLANKECTDYSAGILGVNTSKLRHVKKEEYNYDLL

SEQ ID NO: 41 - DNA, 462 bp, Vibrio ichthyoenteri
atgccacttggttttgcgttgctgcctttaacggcggtggcggaaagtttatgtccgacaaccgaacaagcggtcttttcgtgtgagattggcactaaggcagtggctgcgtgtgtggctgaagatggcaaggtgagttaccgctatggcacgcaaaccaagcttgagttacaactcgatgagccagtgttaagcactggtggttgctctggtggtggaacatctcggcttcgttttgctaacggtgattacagctatatcgtttatgacgtgatgtgtaacgcagaaaaaattggtcctgctcaatggagtaaaaccgattatgccggtttgatggtattgaaaggcaataagttactcgccaataaagagtgcactgactattccgccggaatccttggggttaataccagcaaattacgccatgtgaagaaagaagaatacaattatgatttgctctag

SEQ ID NO: 42 - protein, 148 aa, Salmonella enterica; identical to SEQ ID NO: 33

SEQ ID NO: 43 - DNA, 483 bp, Salmonella enterica; identical to SEQ ID NO: 35

SEQ ID NO: 44 - protein, 163 aa, Salmonella enterica; identical to SEQ ID NO: 36

SEQ ID NO: 45 - DNA, 519 bp, Salmonella enterica; identical to SEQ ID NO: 37

SEQ ID NO: 46 - DNA, 46 bp, Artificial Sequence
  DNA oligonucleotide
gatcctgcaggatgacgatgacaaacaccatcatcaccatcattag

SEQ ID NO: 47 - DNA, 46 bp, Artificial Sequence
  DNA oligonucleotide
agctctaatgatggtgatgatggtgtttgtcatcgtcatcctgcag

SEQ ID NO: 48 - DNA, 45 bp, Artificial Sequence
  DNA oligonucleotide
tttcgaattctagaggaaacaaaaatgggcagggtaatccgtatc

SEQ ID NO: 49 - DNA, 35 bp, Artificial Sequence
  DNA oligonucleotide
ttcccagatctcggccggcatcgggtaaaagatag

SEQ ID NO: 50 - DNA, 32 bp, Artificial Sequence
  DNA oligonucleotide
tttataagcttgctttatgcccgcgcggtgtt

SEQ ID NO: 51 - DNA, 45 bp, Artificial Sequence
  DNA oligonucleotide
tttcgaattctagaggaaacaaaaatgcgtaaattattagtaatt

SEQ ID NO: 52 - DNA, 35 bp, Artificial Sequence
  DNA oligonucleotide
tgttggatccctaaagtattcatcaaatgaacatc

SEQ ID NO: 53 - DNA, 51 bp, Artificial Sequence
  DNA oligonucleotide
tttagaattctaaggagttacatttatgagtaaattattagtaattactgc

(19) United States
(12) Patent Application Publication
(10) Pub. No.: US 2014/0010836 A1
(43) Pub. Date: Jan. 9, 2014

(54) ANTIGENIC GLY1 POLYPEPTIDES

(71) Applicant: Jon Sayers, Chesterfield (GB)

(72) Inventor: Jon Sayers, Chesterfield (GB)

(73) Assignee: University of Sheffield, Sheffield (GB)

(21) Appl. No.: 13/981,492

(22) PCT Filed: Feb. 7, 2012
(86) PCT No.: PCT/GB2012/050259

§ 371 Date: Aug. 29, 2013

(30) Foreign Application Priority Data
Feb. 8, 2011 (GB) 1102091.4

(51) Int. Cl. C07K 14/285

(52) U.S. Cl. CPC C07K 14/285
USPC 424/190.1; 424/234.1

(57) ABSTRACT

We disclose antigenic polypeptides that induce the production of opsonins, in particular opsonic antibodies, and the use of said antigenic polypeptides in vaccines that are protective against bacterial animal pathogens in particular bacterial pathogens of agriculturally important animal species and companion animals and including zoonotic Gram negative bacterial species.

### INTRODUCTION

The disclosure relates to antigenic polypeptides that induce the production of opsonins, in particular opsonic antibodies, and the use of said antigenic polypeptides in vaccines that are protective against bacterial animal pathogens in particular bacterial pathogens of agriculturally important animal species and companion animals and including zoonotic Gram negative bacterial species.

### BACKGROUND TO THE DISCLOSURE

Pathogenic bacteria are a major cause of infectious diseases that affect animals. The control of bacterial infection in agriculturally important animal species is problematic due to the close proximity of animals to each other which can facilitate the dissemination of infection throughout a herd. Herd immunity exists when a larger proportion of animals are immune to a particular infectious agent but can be undermined once a significant number of non-immunized animals are present in the herd. To implement herd immunity it is necessary to continually monitor animals for susceptible members of the herd to control transmission. The control of bacterial transmission is by a number of measures which are labour intensive and expensive to implement and include, quarantine; elimination of the animal reservoir of infection; environmental control [i.e. maintenance of clean water and food supply, hygienic disposal of excrement, air sanitation]; use of antibiotics; use of probiotics to enhance the growth of non-pathogenic bacteria and inhibit the growth of pathogenic bacteria; and active immunization to increase the number of resistant members of a herd. The production of herds that are generally resistant to bacterial infection requires the identification of antigens that form the basis of vaccines which induce immunity.

Furthermore animal species harbour bacterial pathogens that also infect humans. A zoonosis is an infectious disease transmittable between a non-human animal to a human. Examples of zoonotic bacterial infections caused by Gram negative bacteria include brucellosis caused by Brucella spp which is transmitted to humans from infected milk and meat; campylobacteriosis caused by Campylobacter spp; cholera caused by Vibrio cholera; yersinosis caused by Yersina spp from infected uncooked meat or unpasteurized milk; and salmonellosis caused by Salmonella spp from infected meat, in particular pork and eggs.

Many modern vaccines are made from protective antigens of the pathogen, isolated by molecular cloning and purified from the materials that give rise to side-effects. These vaccines are known as ‘subunit vaccines’. The development of subunit vaccines has been the focus of considerable research in recent years. The emergence of new pathogens and the growth of antibiotic resistance have created a need to develop new vaccines and to identify further candidate molecules useful in the development of subunit vaccines. Likewise the discovery of novel vaccine antigens from genomic and proteomic studies is enabling the development of new subunit vaccine candidates, particularly against bacterial pathogens. However, although subunit vaccines tend to avoid the side effects of killed or attenuated pathogen vaccines, their ‘pure’ status means that subunit vaccines do not always have adequate immunogenicity to confer protection.

As mentioned above vaccines induce the production of antibodies and/or cytolytic T cells that target organisms that express the particular inducing antigen. Antigens that may confer protection tend to be those expressed at the cell surface of the pathogen or alternatively secreted into the surrounding environment and therefore accessible to the immune system. Induced antibodies can function in the process known as opsonisation. Opsonisation is a process by which microbial pathogens are targeted for ingestion by phagocytic cells of the immune system. The binding of opsonins attracts phagocytic cells which results in destruction of the bacterial pathogen. Phagocytosis is mediated by macrophages and polymorphic leukocytes and involves the ingestion and digestion of micro-organisms, damaged or dead cells, cell debris, insoluble particles and activated clotting factors. Opsonins are agents which facilitate the phagocytosis of the above foreign bodies. Opsonic antibodies are therefore antibodies which provide the same function. Examples of opsonins are the Fc portion of an antibody or complement component C3.

This disclosure relates to the identification of a class of protective antigen that advantageously, but not exclusively, induces the production of opsonins that target animal [i.e. non-human] bacterial pathogens, for example the cattle/sheep pathogen Manheimia haemolytica and Haemophilus somnus.

The Gly1 antigen is a secreted protein and shown to be essential to the growth of Neisseria meningitidis on haem and haemoglobin. Gly 1 is involved in iron metabolism and provides an essential function since the phenotype deletion mutants in Gly 1 is failure to grow. We disclose vaccine compositions comprising Gly 1 and sequence variants thereof and their use in the prophylactic and therapeutic vaccination of non-human animals.

### STATEMENTS OF INVENTION

According to an aspect of the invention there is provided a vaccine composition comprising a polypeptide isolated from a bacterial animal pathogen wherein said polypeptide has:
i) an amino acid sequence selected from the group consisting of: SEQ ID NO: 1, 2, 5, 6, 9, 10, 13, 14, 17, 18, 21, 22, 25, 26, 29, 30, 33, 38, 40.
ii) an amino acid sequence as defined in i) above and which is modified by addition, deletion or substitution of one or more amino acid residues and which retains or has enhanced haem binding activity and/or reduced haemolytic activity.

A modified polypeptide as herein disclosed may differ in amino acid sequence by one or more substitutions, additions, deletions, truncations that may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and aspartic acid; c) asparagine and glutamine d) arginine and lysine; e) isoleucine, leucine, methionine and valine and f) phenylalanine, tyrosine and tryptophan. Most highly preferred are variants that retain or enhance the same biological function and activity as the reference polypeptide from which it varies.

In one embodiment, the variant polypeptides have at least 35% identity, more preferably at least 40% identity, even more preferably at least 45% identity, still more preferably at least 50%, 60%, 70%, 80%, 90% identity, and most preferably at least 95%, 96%, 97%, 98% or 99% identity with the full length amino acid sequences illustrated herein.

In a preferred embodiment of the invention said antigenic polypeptide comprises or consists of an amino acid sequence as represented in SEQ ID NO: 1, 2, 5, 6, 9, 10, 13, 14, 17, 18, 21, 22, 25, 26, 29, 30, 33, 38 or 40.

In a preferred embodiment of the invention said antigenic polypeptide comprises or consists of an amino acid sequence selected from SEQ ID NO: 1, 2, 5 or 6.

According to a further aspect of the invention there is provided a vaccine composition comprising a nucleic acid molecule comprising a nucleotide sequence that encodes an antigenic polypeptide isolated from a bacterial animal pathogen wherein the nucleic acid molecule:
i) comprises a nucleotide sequence selected from the group consisting of: SEQ ID NO: 3, 4, 7, 8, 11, 12, 15, 16, 19, 20, 23, 24, 27, 28, 31, 32, 34, 39 or 41.
ii) comprises a nucleotide sequence wherein said sequence is degenerate as a result of the genetic code to the nucleotide sequence defined in (i); and
iii) is a nucleic acid molecule the complementary strand of which hybridizes under stringent hybridization conditions to the nucleotide sequence in i) and ii) above wherein said nucleic acid molecule encodes a haem binding protein.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)
Hybridization: 5×SSC at 65° C. for 16 hours
Wash twice: 2×SSC at room temperature (RT) for 15 minutes each
Wash twice: 0.5×SSC at 65° C. for 20 minutes each

High Stringency (Allows Sequences that Share at Least 80% Identity to Hybridize)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each

Low Stringency (Allows Sequences that Share at Least 50% Identity to Hybridize)
Hybridization: 6×SSC at RT to 55° C. for 16-20 hours
Wash at least twice: 2×-3×SSC at RT to 55° C. for 20-30 minutes each.

In a preferred embodiment of the invention said nucleic acid molecule comprises or consists of a nucleotide sequence as represented in SEQ ID NO: 3, 4, 7, 8, 11, 12, 15, 16, 19, 20, 23, 24, 27, 28, 31, 32, 34, 39 or 41.

In a preferred embodiment of the invention said nucleic acid molecule comprises or consists of a nucleotide sequence selected from the group consisting of: SEQ ID NO: 3, 4, 7 or 8.

In a preferred embodiment of the invention said nucleic acid molecule comprises a transcription cassette comprising: a nucleic acid molecule that encodes said antigenic polypeptide operably linked to a promoter adapted for transcription of the nucleic acid molecule associated therewith.

In a preferred embodiment of the invention said promoter is a constitutive promoter.

In an alternative preferred embodiment of the invention said promoter is a regulatable promoter; preferably an inducible promoter and/or a tissue/cell specific promoter.

“Promoter” is an art recognised term and, for the sake of clarity, includes the following features which are provided by example only. Enhancer elements are cis acting nucleic acid sequences often found 5′ to the transcription initiation site of a gene (enhancers can also be found 3′ to a gene sequence or even located in intronic sequences). Enhancers function to increase the rate of transcription of the gene to which the enhancer is linked. Enhancer activity is responsive to trans acting transcription factors which have been shown to bind specifically to enhancer elements. The binding/activity of transcription factors (please see Eukaryotic Transcription Factors, by David S Latchman, Academic Press Ltd, San Diego) is responsive to a number of physiological/environmental cues. Promoter elements also include so called TATA box and RNA polymerase initiation selection sequences which function to select a site of transcription initiation. These sequences also bind polypeptides which function, inter alia, to facilitate transcription initiation selection by RNA polymerase.

In a preferred embodiment of the invention said promoter is a skeletal muscle specific promoter.

Muscle specific promoters are known in the art. For example, WO0009689 discloses a striated muscle preferentially expressed gene and cognate promoter, the SPEG gene.

EP1072680 discloses the regulatory region of the myostatin gene. The gene shows a predominantly muscle specfic pattern of gene expression. U.S. Pat. No. 5,795,872 discloses the use of the creatine kinase promoter to achieve high levels of expression of foreign proteins in muscle tissue. The muscle specific gene Myo D also shows a pattern of expression restricted to myoblasts. Further examples are disclosed in WO03/074711.

Preferably said consititutive promoter is selected from the group consisting of: Cytomegalovirus (CMV) promoter, β-globin RSV enhancer/promoter phosphoglycerate kinase (mouse PGK) promoter, alpha-actin promoter, SV40 promoter EF-1α promoter, ubiquitin promoter, transcription factor A (Tfam) promoter.

In a preferred embodiment of the invention said nucleic acid molecule is part of a vector.

In a preferred embodiment of the invention said vector is an expression vector adapted for expression of said nucleic acid molecule encoding said antigenic polypeptide according to the invention; preferably said nucleic acid molecule is operably linked to at least one promoter sequence.

There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, NY and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc. (1994).

The use of viruses or “viral vectors” as therapeutic agents is well known in the art. Additionally, a number of viruses are commonly used as vectors for the delivery of exogenous genes. Commonly employed vectors include recombinantly modified enveloped or non-enveloped DNA and RNA viruses, preferably selected from retro viridae baculoviridiae, parvoviridiae, picornoviridiae, herpesveridiae, poxyiridae, adenoviridiae, or picomnaviridiae. Chimeric vectors may also be employed which exploit advantageous elements of each of the parent vector properties (See e.g., Feng, et al. (1997) Nature Biotechnology 15:866-870). Such viral vectors may be wild-type or may be modified by recombinant DNA techniques to be replication deficient, conditionally replicating or replication competent. Preferred vectors are derived from retroviral genomes [e.g. lentivirus] or are adenoviral based.

Viral vectors may be conditionally replicating or replication competent. Conditionally replicating viral vectors are used to achieve selective expression in particular cell types while avoiding untoward broad spectrum infection. Examples of conditionally replicating vectors are described in Pennisi, E. (1996) Science 274:342-343; Russell, and S. J. (1994) Eur. J. of Cancer 30A(8):1165-1171. Additional examples of selectively replicating vectors include those vectors wherein a gene essential for replication of the virus is under control of a promoter which is active only in a particular cell type or cell state such that in the absence of expression of such gene, the virus will not replicate. Examples of such vectors are described in Henderson, et al., U.S. Pat. No. 5,698,443 issued Dec. 16, 1997 and Henderson, et al.; U.S. Pat. No. 5,871,726 issued Feb. 16, 1999 the entire teachings of which are herein incorporated by reference.

Additionally, the viral genome may be modified to include inducible promoters which achieve replication or expression only under certain conditions. Examples of inducible promoters are known in the scientific literature (See, e.g. Yoshida and Hamada (1997) Biochem. Biophys. Res. Comm. 230:426-430; lida, et al. (1996) J. Virol. 70 (9):6054-6059; Hwang, et al. (1997) J. Virol 71 (9):7128-7131; Lee, et al. (1997) Mol. Cell. Biol. 17 (9):5097-5105; and Dreher, et al. (1997) J. Biol. Chem. 272 (46); 29364-29371.

In a preferred embodiment of the invention said polypeptide or said nucleic acid molecule is isolated from a Gram negative bacterial animal pathogen.

In a preferred embodiment of the invention said polypeptide or said nucleic acid molecule is isolated from a Gram negative zoonotic bacterial animal pathogen.

In a preferred embodiment of the invention said bacterial animal pathogen is selected from the genus group consisting of: Mannheimia spp, Actinobacillus spp, Pasteurella spp, Haemophilus spp, Edwardsiella spp or Avibacterium spp [for example A. paragallinarum].

Additional bacterial pathogens include zoonotic species selected from Brucella spp, Campylobacter spp, Vibrio spp [for example V. ichthyoenteri], Yersina spp, and Salmonella spp [for example Salmonella enterica].

In a preferred embodiment of the invention said composition further comprises an adjuvant or carrier.

Adjuvants (immune potentiators or immunomodulators) have been used for decades to improve the immune response to vaccine antigens. The incorporation of adjuvants into vaccine formulations is aimed at enhancing, accelerating and prolonging the specific immune response to vaccine antigens. Advantages of adjuvants include the enhancement of the immunogenicity of weaker antigens, the reduction of the antigen amount needed for a successful immunisation, the reduction of the frequency of booster immunisations needed and an improved immune response in elderly and immunocompromised vaccinees. Selectively, adjuvants can also be employed to optimise a desired immune response, e.g. with respect to immunoglobulin classes and induction of cytotoxic or helper T lymphocyte responses. In addition, certain adjuvants can be used to promote antibody responses at mucosal surfaces. Aluminium hydroxide and aluminium or calcium phosphate has been used routinely in human vaccines. More recently, antigens incorporated into IRIV's (immunostimulating reconstituted influenza virosomes) and vaccines containing the emulsion-based adjuvant MF59 have been licensed in countries. Adjuvants can be classified according to their source, mechanism of action and physical or chemical properties. The most commonly described adjuvant classes are gel-type, microbial, oil-emulsion and emulsifier-based, particulate, synthetic and cytokines. More than one adjuvant may be present in the final vaccine product. They may be combined together with a single antigen or all antigens present in the vaccine, or each adjuvant may be combined with one particular antigen. The origin and nature of the adjuvants currently being used or developed is highly diverse. For example, aluminium based adjuvants consist of simple inorganic compounds, PLG is a polymeric carbohydrate, virosomes can be derived from disparate viral particles, MDP is derived from bacterial cell walls; saponins are of plant origin, squalene is derived from shark liver and recombinant endogenous immunomodulators are derived from recombinant bacterial, yeast or mammalian cells.

There are several adjuvants licensed for veterinary vaccines, such as mineral oil emulsions that are too reactive for human use. Similarly, complete Freund's adjuvant, although being one of the most powerful adjuvants known, is not suitable for human use.

A carrier is an immunogenic molecule which, when bound to a second molecule augments immune responses to the latter. The term carrier is construed in the following manner. A carrier is an immunogenic molecule which, when bound to a second molecule augments immune responses to the latter. Some antigens are not intrinsically immunogenic yet may be capable of generating antibody responses when associated with a foreign protein molecule such as keyhole-limpet haemocyanin or tetanus toxoid. Such antigens contain B-cell epitopes but no T cell epitopes. The protein moiety of such a conjugate (the “carrier” protein) provides T-cell epitopes which stimulate helper T-cells that in turn stimulate antigen-specific B-cells to differentiate into plasma cells and produce antibody against the antigen.

In a preferred embodiment of the invention said adjuvant is selected from the group consisting of aluminium hydroxide, aluminium or calcium phosphate.

In a preferred embodiment of the invention said adjuvant is selected from the group consisting of: cytokines selected from the group consisting of GMCSF, interferon gamma, interferon alpha, interferon beta, interleukin 12, interleukin 23, interleukin 17, interleukin 2, interleukin 1, TGF, TNFα, and TNFβ.

In a further alternative embodiment of the invention said adjuvant is a TLR agonist such as CpG oligonucleotides, flagellin, monophosphoryl lipid A, poly I:C and derivatives thereof.

In a preferred embodiment of the invention said adjuvant is a bacterial cell wall derivative such as muramyl dipeptide (MDP) and/or trehalose dicorynomycolate (TDM).

According to a further aspect of the invention there is provided an antigenic polypeptide isolated from a bacterial animal pathogen comprising or consisting of an amino acid sequence selected from the group consisting of the amino acid sequence selected from the group consisting of SEQ ID NO: 1, 2, 5, 6, 9, 10, 13, 14, 17, 18, 21, 22, 25, 26, 29, 30, 33, 38 or 40 for use in the production of an opsonin[s].

In a preferred embodiment of the invention said antigenic polypeptide comprises or consists of SEQ ID NO: 1, 2, 5 or 6.

In an alternative preferred embodiment of the invention said antigenic polypeptide is encoded by a nucleic acid molecule comprising or consisting of the nucleotide sequence selected from the group consisting of SEQ ID NO: 3, 4, 7, 8, 11, 12, 15, 16, 19, 20, 23, 24, 27, 28, 31, 32, 34, 39 or 41.

Preferably said nucleic acid molecule comprises SEQ ID NO: 3, 4, 7 or 8.

In a preferred embodiment of the invention said opsonin is an antibody.

According to an aspect of the invention there is provided a method for immunizing a non-human animal against a pathogenic bacteria comprising:
i) administering an effective amount of a dose of a vaccine composition according to the invention to a non-human animal subject to induce protective immunity; optionally
ii) administering one or more further dosages of vaccine to said subject sufficient to induce protective immunity.

According to a further aspect of the invention there is provided a vaccine composition according to the invention for use in the treatment of Gram negative bacterial pathogenic infection in a non-human animal subject.

According to a further aspect of the invention there is provided a method for the production of an opsonin to an antigen derived from a non-human animal bacterial pathogen comprising:
i) providing a vaccine composition according to the invention;
ii) administering an effective amount of said composition to a non-human animal subject sufficient to induce opsonin production.

The vaccine compositions of the invention can be administered by any conventional route, including injection. The administration may be, for example, intravenous, intraperitoneal, intramuscular, intracavity, subcutaneous, or intradermally. The vaccine compositions of the invention are administered in effective amounts. An “effective amount” is that amount of a vaccine composition that alone or together with further doses, produces the desired response. In the case of treating a particular bacterial disease the desired response is providing protection when challenged by an infective agent.

It is generally preferred that a maximum dose of the individual components or combinations thereof be used sufficient to provoke immunity; that is, the highest safe dose according to sound veterinary judgment. The doses of vaccine administered to an animal subject can be chosen in accordance with different parameters, in particular in accordance with the mode of administration used and the state of the animal subject. In the event that a response in a subject is insufficient at the initial doses applied, higher doses (or effectively higher doses by a different, more localized delivery route) may be employed to the extent that tolerance permits. In general, doses of vaccine are formulated and administered in effective immunizing doses according to any standard procedure in the art. Other protocols for the administration of the vaccine compositions will be known to one of ordinary skill in the art, in which the dose amount, schedule of injections, sites of injections, mode of administration and the like vary from the foregoing.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

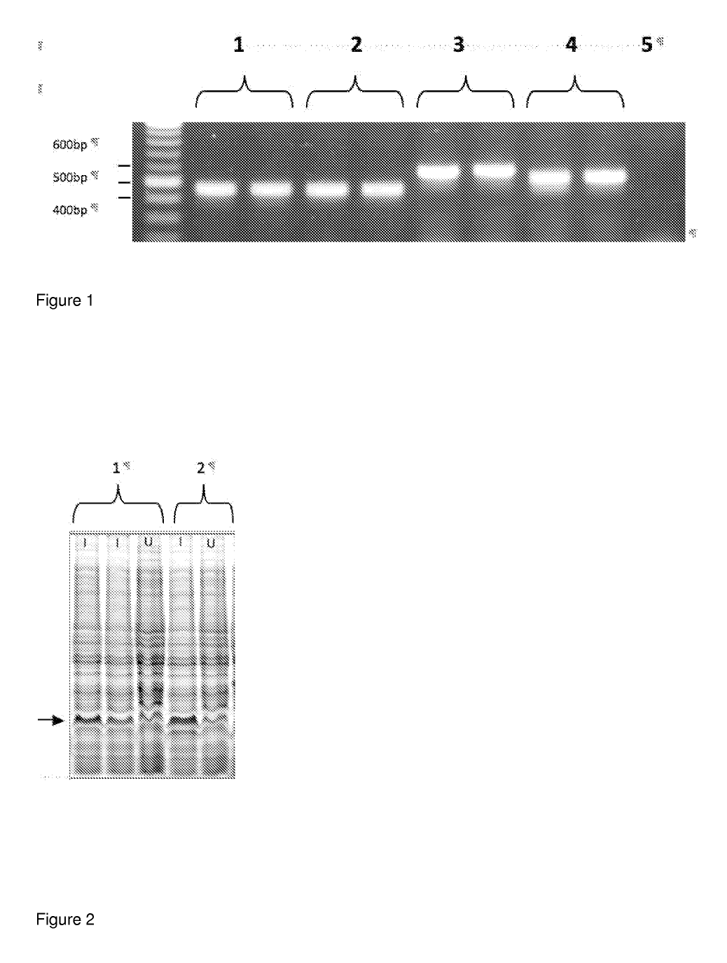

FIG. 1 illustrates and ethidium bromide stained agarose gel showing the PCR-based amplification of recombinant Gly1 genes from Mannheimia haemolytica and Edwardsiella ictaluri. The gel shows duplicate samples of 1) M. haemolytica with primersGly1_man.f and Gly1_Man.r, 2) M. haemolytica with primers Gly1_man.f and Gly1_man.H6.r, 3) Edwardsiella ictaluri with primersGly1_cat.f and Gly1_cat.r, 4) E. ictaluri with Gly1_cat.f and Gly1_cat.h6.r. Lane 5 shows a negative control.

FIG. 2 illustrates a Coomassie stained SDS-PAGE analysis of expression of C-terminal histidine tag recombinant Gly1ORF1 homologs. U—uninduced and I—induced cell pellet, 1—M. haemolytica S2 C-his Gly1ORF1 homolog, 2—M. haemolytica C-his Gly1ORF1 homolog, An arrow shows a band corresponding to induced protein;

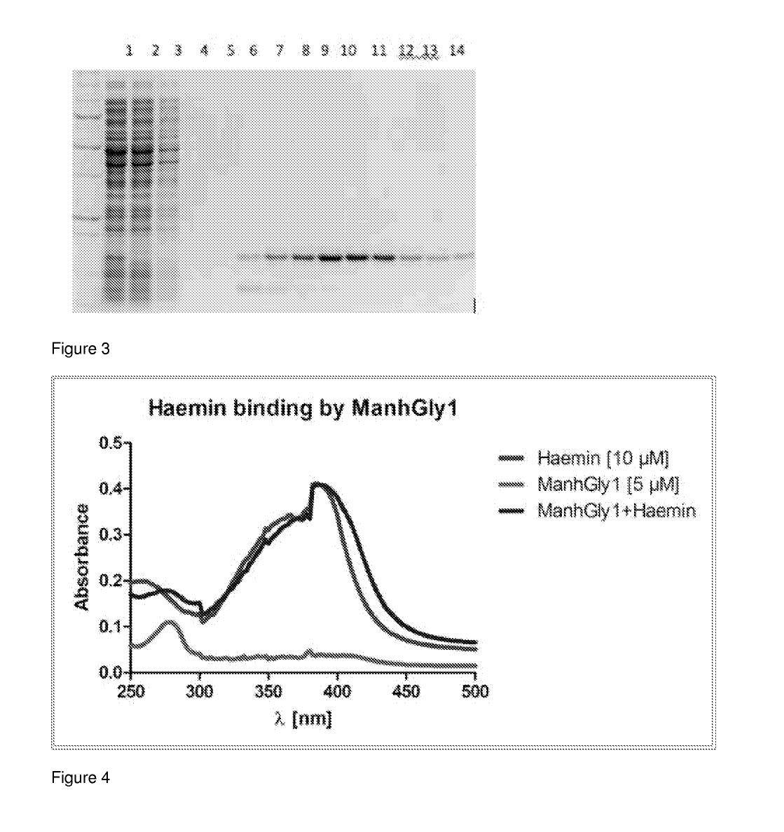

FIG. 3 illustrates a Coomassie stained SDS-PAGE analysis demonstrating purification of M. haemolytica Gly1ORF1 homolog with C-terminal his-tag on Ni-chelate column. 1—loaded on the Ni-column soluble fraction of the cell lysate, 2—flow-through, 3—wash, lanes 4-14—elution fractions;

FIG. 4 Changes in haemin spectrum after addition of ManhGly1. Manheimia Gly1 changes the hemin visible spectrum indicating that these molecules interact;

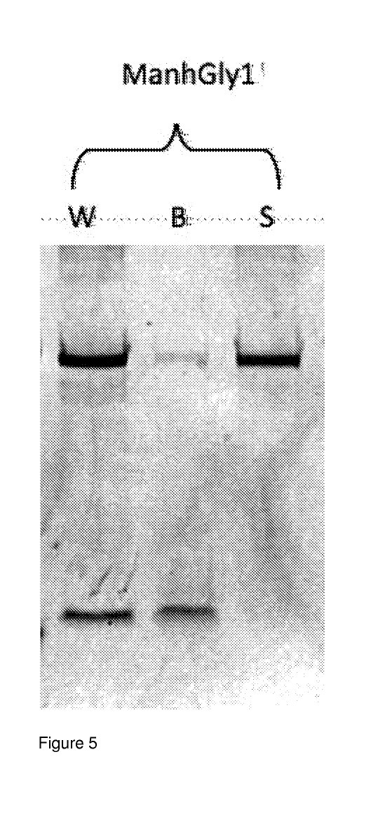

FIG. 5 illustrates Coomassie stained SDS-PAGE-analysis of hemin-agarose bead pull-down assay showing selective binding of ManhGly1. W—ManhGly1 with BSA before incubation with haemin beads, B—beads incubated with the proteins mixture shown in W, washed and boiled in SDS, S—supernatant W incubated with ManhGly1 after pelleting the beads. An arrow shows a band corresponding to BSA. The hemin-agarose beads selectively remove ManhGly1 from the mixture; and

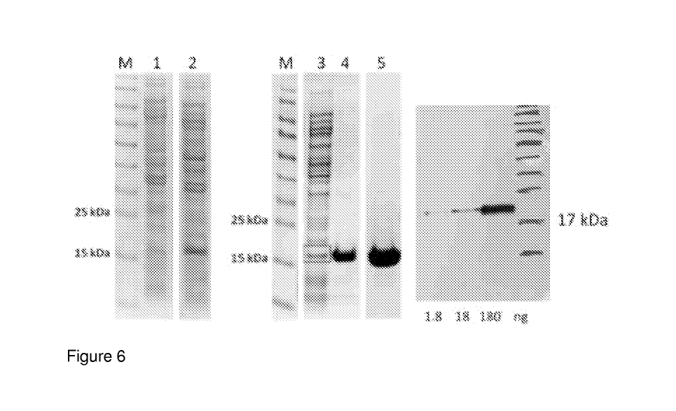

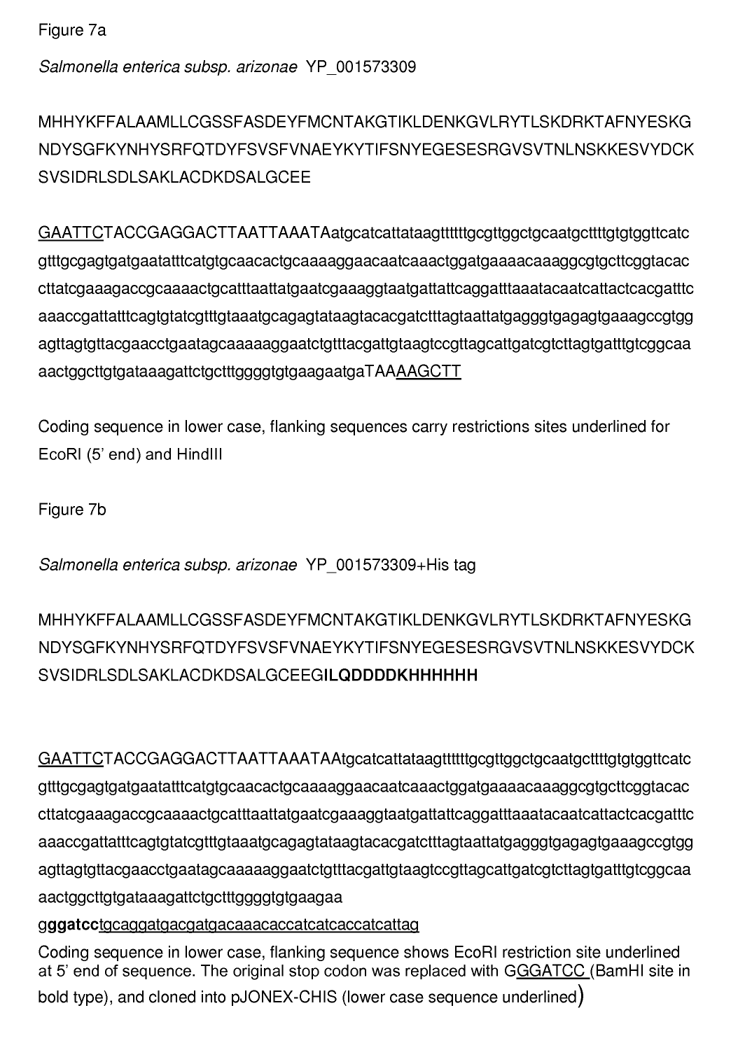

FIG. 6 illustrates the cloning, expression and westernblotting of Salmonella enterica Gly1 homologue. SDS-PAGE gel showing protein size markers (M), total protein from M72 cells carrying recombinant gene for C-his tagged SalGly1 before (lane 1) and after (lane 2) induction. After lysis, soluble protein (Lane 3), protein was purified by nikel chelate (Lane 4) and ion exchange chromatography (Lane 5). The right hand panel shows the results of a western blot using the indicated amounts of SalGly1 protein with primary antisera raised in rats at a dilution of 1:5000.

### MATERIALS AND METHODS

Cloning:

The plasmid pJONEX4 (J. R. Sayers, F. Eckstein, Nucleic Acids Res. 19, 4127 (1991) was digested with BamHI and HindIII and ligated with a duplex DNA consisting of two oligonucleotides (C1 5′ GATCCTGCAGGATGACGATGACAAACACCATCATCACCATCATTAG and C2 5′ AGCTCTAATGATGGTGATGATGGTGTTTGTCATCGTCATCCTGCAG) to create a plasmid designated pJONEX-CHIS which was transfected into competent E. coli cells and the progeny plasmids were characterized by DNA sequencing.

Edwardsiella ictaluri genomic DNA was used as a template with the following primers: Gly1_cat.f (5′-TTTCGAATTCTAGAGGAAACAAAAATGGGCAGGGTAATCCGTATC) with either Gly1_cat.rH6 (5′-TTCCCAGATCTCGGCCGGCATCGGGTAAAAGATAG) or Gly1_cat.r (5′-TTTATAAGCTTGCTTTATGCCCGCGCGGTGTT). Gly1_cat.f contains an EcoRI site, Gly1_cat.rH6 contains a BglII site for cloning into the vector pJONEX-CHIS described above using the compatible BamHI in the vector. Gly1_cat.r contains a HindIII site.

Mannheimia haemolytica genomic DNA (Intervet Innovation GmbH, Schwabenheim, Germany) was used as a template for PCR. Primers Gly1_man.f (TTTCGAATTCTAGAGGAAACAAAAATGCGTAAATTATTAGTAATT) and Gly1_man.h6.r (TGTTGGATCCCTAAAGTATTCATCAAATGAACATC) were used to produce a PCR product encoding the a C-terminal his-tagged M. haemolytica gly1 (ManhGly1 C6H-tag) by standard methods. A variant with codon 2 (Arg) replaced with a serine codon was constructed similarly using primer Gly1_ManS2.f (TTTAGAATTCTAAGGAGTTACATTTATGAGTAAATTATTAGTAATTACTGC) with primer Gly1_man.h6.r to create an alternative (more highly expressed) version of the protein.

The amplified products were subjected to digestion with restriction endonucleases EcoRI and BamHI and ligated into pJONEX4 (cut with the same restriction enzymes) to generate plasmids carrying the recombinant genes. This results in an in-frame fusion of the Gly1-coding region with a C-terminal enterokinase recognition site, six histidine residues, and stop codon The resulting plasmids were designated as pJONGLY-Man1 and pJONGLYMan2, expressing the Arg2 or Ser2 variant proteins respectively. The newly constructed DNA was transfected into M72(λ) at 28° C. on LB-ampicillin plates. The resulting recombinant constructs were sequenced (Core Genomics Facility, University of Sheffield Medical School) using standard M13 forward and reverse primers.

Protein Production:

An overnight culture (100 mL) of M72(λ) carrying the required plasmid was grown in 5YT media containing 100 □g ml⁻¹ carbenicillin was inoculated into a 2 L fermenter containing 1.5 L of 5YT/carbenicillin media, incubated at 30° C., stirred at 750 rpm and provided with an air supply of 2 L min⁻¹. At mid-log phase the temperature was increased to 42° C. for 3 hr. The cells were then removed from the spent supernatant by centrifugation at 10,000×g for 20 min at room temperature. A similar approach was used for the other gly1 homologues.

Protein purification: Proteins in the supernatant were precipitated by addition of ammonium sulphate to 3 M final concentration and were then recovered by centrifugation at 40,000×g for 20 min at 10° C. The protein pelleted was resuspended in phosphate buffered saline or Tris-buffered saline pH8, dialyzed extensively, and applied to a nickel-chelate affinity chromatography column. The column was then washed and eluted with either a gradient of imidazole or an acid acid gradient according to standard protocol, purification was monitored by SDS PAGE. An alternative method made use of the cell pellet which contained expressed fusion protein. The fermenter cell pellet was resuspended with 5 ml of resuspension buffer (50 mM Tris HCl [pH 8.2], 2 mM EDTA, 200 mM NaCl, 1 mM DTT and 5% (v/v) glycerol) per gram of pellet. In order to lyse the cells, lysozyme (Sigma) was added to final concentration of 200 μg ml⁻¹ and stirred on ice until the mixture became viscous (approximately 30 min). Phenylmethylsulfonylfluoride (PMSF) was then added to a final concentration of 23 μg ml⁻¹ to inhibit the activity of serine proteases present in the lysate, which may have degraded Gly1ORF1. Sodium deoxycholate was subsequently added to a final concentration of 500 μg ml⁻¹ and the mixture was stirred on ice for 20 min.

In order to fracture genomic DNA released from lysis of the bacterial cells sonication was performed. The amplitude was set to 20-30% and three rounds of sonication were carried out for 15 seconds on ice using a Vibracell™ VCX400 sonicator (Sonics and Materials Inc, Danbury, Conn., USA). Following stirring at 4° C. for 40 min, sonication was repeated as above until the sample was no longer viscous due to the shearing of genomic DNA into smaller fragments. The sample was then centrifuged at 40,000×g for 30 min to pellet the insoluble portion of the lysate. The supernatant was then adjusted to 3 M in ammonium sulphate and proteins were precipitated by centrifugation at 43,000×g for 30 min at 10° C. The protein was then purified as described above.

Alternatively, proteins were extracted from the cell pellet by resuspending in packed cells in 50 mM Tris HCl, pH 8, 200 mM NaCl (10 ml per g of packed cells) then solid guanidinium hydrochloride with stirring until the suspension cleared. The viscosity was reduced by sonication and debris removed by centrifugation at 43,000×g for 30 min at room temperature. The supernatant was then removed, diluted to approx. 3 M in guanidinium hydrochloride, centrifuged as before and applied to a nickel chelate affinity chromatography column. The guanidinium hydrochloride was removed by washing the column in 10 volumes of compatible buffer, followed by washing with either an acid or imidazole gradient to effect elution.

Haem (Hemin) Binding Assays:

The change in the UV-visible spectrum of free and Gly1 bound hemin was examined by spectroscopy and using hemin-agarose beads as an affinity matrix to pull down Gly1 proteins from a mixture of Gly1 with carrier BSA.

Production of Antibodies:

Recombinant Gly1 protein samples can be used to immunize rabbits using standard protocols. These antibodies can then be used in serum bacteriocidal antibody assay as outlined: Dilutions of the target organism are made such that approx. 1500 c.f.u are incubated in PBS containing 1% FCS in the presence of different dilutions of anti-Gly1 antibodies (1/10,-1/10,000) and appropriate serum which acts as the source of complement in 96 well plates. The controls without antibodies and or the serum should be carried out in parallel. After an hour of incubation at 37° C. and 5% CO₂ a 20 μl sample from each reaction should be plated onto appropriate selective media plates and grown for 16 hours at 37° C. (with 5% CO₂ if appropriate). Following the incubation, the viable cells are enumerated. In this way it can be determined whether antibodies directed against a particular bacterial Gly1 protein are capable of directing serum bacteriocidal activity in an target animal of choice.

Construction and Synthesis of a Salmonella enterica Gly 1 Homologue

A synthetic DNA encoding the Salmonella enterica subsp. arizonae serovar amino acid sequence (YP_{—}001573309), a Neisseria meningitidis GLY1ORF1 homologue, was constructed by gene synthesis (Eurof ins MWG) and cloned into the pJONEX4 plasmid via flanking EcoRI and HindIII sites. Similarly, a DNA construct in which the stop codon was replaced with a BamHI site so as to allow expression of YP_{—}001573309 as a fusion protein sequence in frame fusion with a C-terminal six histidine tag was prepared and subcloned into pJONEX-CHIS. The recombinant plasmids were used to produce tagged and untagged S. enterica Gly1 homologue (SalGLY1).

Anti-sera. The SalGLY1 protein was used to raise antisera in rats (BioServ Ltd). The sera was then used in western blotting; see FIG. 6.

### EXAMPLES

Recombinant Gly1 genes can be readily amplified by PCR as exemplified by the DNA fragments obtained from PCR of appropriate primers with genomic DNA from Mannheimia haemolytica and Edwardsiella ictaluri. This is shown in FIG. 1.

Recombinant Mannheimia haemolytica Gly1 protein was readily produced in E. coli as shown in FIG. 2. The protein can be purified using standard methods as shown in FIG. 3.

The Gly1 protein from Mannheimia haemolytica caused as shift in the visible spectrum of hemin as demonstrated in FIG. 4, indicating that they bind one another.

The interaction of a Gly1 protein with haem (hemin) can be confirmed using pull-down assays with hemin-agarose beads as shown in FIG. 5. By way of example, this shows that haem binds M. haemolytica Gly1 protein selectively from a mixture of Gly1 and bovine serum albumin.

FIG. 6 illustrates a SDS-PAGE gel showing protein size markers (M), total protein from M72 cells carrying recombinant gene for C-his tagged SalGly1 before (lane 1) and after (lane 2) induction. After lysis, soluble protein (Lane 3), protein was purified by nickel chelate (Lane 4) and ion exchange chromatography (Lane 5). The right hand panel shows the results of a western blot using the indicated amounts of SalGly1 protein with primary antisera raised in rats at a dilution of 1:5000.

1. A vaccine composition comprising an antigenic polypeptide wherein said antigenic polypeptide is isolated from a bacterial animal pathogen and comprises:
i) an amino acid sequence selected from the group consisting of: SEQ ID NO: 1, 2, 5, 6, 9, 10, 13, 14, 17, 18, 21, 22, 25, 26, 29, 30, 33, 38, and 40; or
ii) an amino acid sequence as defined in i) above and which is modified by addition, deletion or substitution of one or more amino acid residues and which retains or has enhanced haem binding activity and/or reduced haemolytic activity.

2. The vaccine composition according to claim 1, wherein said antigenic polypeptide comprises or consists of an amino acid sequence as represented in SEQ ID NO: 1, 2, 5, 6, 9, 10, 13, 14, 17, 18, 21, 22, 25, 26, 29, 30, 33, 38 or 40.

3. The vaccine composition according to claim 1 wherein said antigenic polypeptide comprises or consists of an amino acid sequence shown in SEQ ID NO: 1, 2, 5 or 6.

4. A vaccine composition comprising a nucleic acid molecule that encodes an antigenic polypeptide isolated from an animal pathogen, wherein the nucleic acid molecule comprises:
i) a nucleotide sequence selected from the group consisting of: SEQ ID NO: 3, 4, 7, 8, 11, 12, 15, 16, 19, 20, 23, 24, 27, 28, 31, 32, 34, 39, and 41;
ii) a nucleotide sequence degenerate as a result of the genetic code to the nucleotide sequence defined in (i); or
iii) a complementary strand to the nucleotide sequence in i) or ii) and which hybridizes under stringent hybridization conditions to the nucleotide sequence in i) and ii) above wherein said nucleic acid molecule encodes a haem binding protein.

5. The vaccine composition according to claim 4, wherein said nucleic acid molecule comprises or consists of a nucleotide sequence as represented in SEQ ID NO: 3, 4, 7, 8, 11, 12, 15, 16, 19, 20, 23, 24, 27, 28, 31, 32, 34, 39 or 41.

6. The vaccine composition according to claim 4, wherein said nucleic acid molecule comprises or consists of a nucleotide sequence shown in SEQ ID NO: 3, 4, 7 or 8.

7. The vaccine composition according to claim 4, wherein said nucleic acid molecule comprises a transcription cassette comprising: the nucleic acid molecule that encodes said antigenic polypeptide operably linked to a promoter adapted for transcription of the nucleic acid molecule that encodes said antigenic polypeptide.

8. The vaccine composition according to claim 7, wherein said nucleic acid molecule is part of a vector.

9. The vaccine composition according to claim 1, wherein said antigenic polypeptide is isolated from a Gram negative bacterial animal pathogen.

10. The vaccine composition according to claim 9, wherein said antigenic polypeptide is isolated from a Gram negative zoonotic bacterial animal pathogen.

11. The vaccine composition according to claim 10 wherein said bacterial animal pathogen is selected from the genus group consisting of: Mannheimia spp, Actinobacillus spp, Pasteurella spp, Haemophilus spp and Edwardsiella spp.

12. The vaccine composition according to claim 10, wherein said bacterial animal pathogen is selected from the group consisting of: Brucella spp, Campylobacter spp, Vibrio spp, Yersina spp Salmonella spp, and Avibacterium spp.

13. The vaccine composition according to claim 1, wherein said composition further comprises an adjuvant or carrier.

14-18. (canceled)

19. A method for immunizing a non-human animal against a pathogenic non human bacterial species comprising:
i) administering an effective amount of a dose of the vaccine composition of claim 1 to a non-human animal subject to induce protective immunity; and optionally
ii) administering one or more further dosages of the vaccine composition to said subject sufficient to induce protective immunity.

20. (canceled)

21. A method for the production of an opsonin to an antigen isolated from a non-human animal bacterial pathogen comprising:
i) providing the vaccine composition of claim 1; and
ii) administering an effective amount of said vaccine composition to a non-human animal subject sufficient to induce opsonin production.

22. A method for treating a Gram negative bacterial pathogenic infection in a non-human animal subject, comprising:
administering an effective amount of the vaccine composition of claim 1 to the non-human animal subject sufficient to treat the Gram negative bacterial pathogenic infection.
